(19) United States
(12) Patent Application Publication
(10) Pub. No.: US 2008/0095789 A1
(43) Pub. Date: Apr. 24, 2008

(54) Vaccine

(76) Inventors: Rolf Kiessling, Bromma (SE); Lars Holmgren, Bromma (SE)

Correspondence Address: KENYON & KENYON LLP, ONE BROADWAY, NEW YORK, NY, 10004, US

(21) Appl. No.: 10/583,950

(22) PCT Filed: Dec. 20, 2004
(86) PCT No.: PCT/EP04/14573

§ 371 Date: Aug. 22, 2007

(30) Foreign Application Priority Data
Dec. 20, 2003 (GB) 0330079.5

(51) Int. Cl. A61K 39/00

(52) U.S. Cl. 424/185.1

(57) ABSTRACT

A vaccine for preventing formation of blood vessels, for example neoangiogenesis, as well as repressing existing vascularization associated with tumors and other diseases, comprising angiomotin, for example as a whole molecule or fragment thereof or encoded as a gene or mRNA, or administered as dendritic cells (DC cells) expressing angiomotin, which may be utilized to generate immune responses to the angiomotin molecule. Inhibition of angiogenesis and delay in tumor outgrowth in animal models of cancer is achieved.

### FIELD OF INVENTION

The present invention relates to prophylactic or therapeutic treatments for hindering blood vessel formation, for example for hindering tumour growth, retinal disease, atherosclerosis, endometriosis, rheumatoid arthritis and inflammatory conditions.

### BACKGROUND OF THE INVENTION

A vaccine is a preparation derived from a disease-causing agent or its components which is administered to stimulate an immune response that will protect a person from illness due to that agent. A therapeutic (treatment) vaccine is given after onset of the disease and is intended to reduce or arrest disease progression. A preventive (prophylactic) vaccine is intended to prevent initial disease onset. Agents used in vaccines may, for example, be whole-killed (inactive), live-attenuated (weakened) pathogenic organisms or artificially manufactured.

Vaccines mediate their effect by stimulating the immune system of the host to specifically generate antibodies and/or immune cells (cytotoxic T-cells) against the principal target. These targets are known as “antigens”. Stimulation of immune responses against the target antigen results in the immune-mediated destruction and elimination of the disease agent residing in the body of the immunized host. Once stimulated, the immune system also maintains surveillance against subsequent infection or development of disease by the targeted agent. Thus vaccines are an effective way of controlling existing disease as well as inhibiting its exacerbation or recurrence in the future.

There are a variety of methods for vaccinating against a target antigen. Some of these include injecting a whole protein; a synthetic peptide corresponding to a fragment of the protein; and/or DNA/RNA sequences encoding the protein. Delivery vehicles for delivering DNA and RNA include altered viruses expressing the gene, naked DNA/RNA or recombinant plasmids that express the antigen in conjunction with other immunostimulatory agents such as cytokines or growth factors.

Vasculogenesis is the differentiation of stem cells into endothelial cells which then form blood vessels. Angiogenesis is the formation of blood vessels from preexisting ones. Terms such as blood vessel formation, neovascularization and vascularization covers both vasculogenesis and angiogenesis.

Angiogenesis (an example of blood vessel formation) is the formation of new capillary blood vessels by a process of sprouting from pre-existing vessels and occurs during development as well as in a number of physiological and pathological settings (Folkman J. Nature Medicine 1995). Formation of new blood vessels by the process of angiogenesis involves a complex series of events including endothelial cell proliferation, migration, interaction and adhesion to form cords and tubes, and finally maturation. Physiologically, angiogenesis is necessary for tissue growth, wound healing, and female reproductive function and is a component of pathological processes such as retinal disease, atherosclerosis, endometriosis, rheumatoid arthritis and inflammatory conditions. However, much of the longstanding interest in angiogenesis comes from the notion that for solid tumors to grow beyond a critical size, they must recruit endothelial cells from the surrounding stroma to form their own endogenous microcirculation. In order to promote neo-vascularisation, tumors release variety of factors that stimulate proliferation and migration of endothelial cells. Such factors include Vascular Endothelial Cell Growth factor (VEGF) and basic Fibroblast Growth Factor (bFGF), interleukin-8 (IL-8) placental growth factor, and thymidine phosphorylase (platelet-derived endothelial cell growth factor, Relf M et al Cancer Research 1997). Therefore, much effort has been dedicated to finding molecules that interfere with these signaling pathways and thereby block tumor angiogenesis.

Targeting angiogenesis has potentially several advantages compared to traditional oncolytic therapy. The most prominent being that all solid tumors are angiogenesis-dependent, the target endothelial cells are readily accessible for therapy, is genomically stable and less prone to generate resistance to therapy. One of the obvious disadvantages of targeting cancer cell expressed protein is the genetic variability and a large selection pressure due to rapid cell growth and division, which often renders such drugs ineffective due to resistance mechanisms and the onset and use of alternative pathways.

Angiogenesis inhibition also is showing early promise with diabetic retinopathy and macular degeneration, which both result from an overgrowth of ocular blood vessels. In these disorders, the vessels interfere with normal structures in the eye, or they block light from reaching the back of the eye. The new blood vessels are themselves the primary pathology, and stopping blood vessel growth could prevent blindness.

A large number of naturally occurring angiogenic inhibitors have been identified such as Angiostatin (plasminogen fragment) Anti-angiogenic anti-thrombin III, Endostatin (collagen XVIII fragment), Interferon alpha/beta/gamma, Prolactin 16 kD fragment and Thrombospondin-1 (TSP-1) which show varying degree of effect in vitro and in vivo models. These inhibitors target endothelial cells and inhibit angiogenesis. The observed inhibition of e.g. Angiostatin, is independent of which angiogenic factor the endothelial cells are stimulated by (Eriksson et al FEBS L. 2003). This is in contrast to agents such as antibodies that bind to VEGF or low molecular compounds that inhibit VEGF-receptor kinase activity. Most tumors express a variety of angiogenic factors indicating that targeting one single angiogenesis pathway is not enough for inhibiting tumor expansion. Thus, therapies that target directly the endothelial cells have a potential to circumvent the problem of angiogenesis being controlled by a plurality of tumor-derived factors. However, on the other hand, such therapies have to deal with the problem of being able to target specifically endothelial cells that are involved in the process of neo-vascularisation while sparing mature blood vessels.

Angiomotin was identified by its binding to another molecule (angiostatin) which is also involved in angiogenesis. (Troyanovsky et al., J. Cell. Biol. 2001; WO 99/66038). Real time PCR analysis of the expression pattern of angiomotin in primary cells as well as in cell-lines have shown that angiomotin is predominantly expressed in endothelial cells. In vivo mapping of angiomotin has revealed expression in angiogenic tissues such as the human placenta as well as tumor tissues. These data suggest that angiomotin is upregulated in endothelial cells during angiogenesis. WO 99/66038 discusses angiomotin and its use as, for example, a drug screening target.

The invention provides the use of vaccines corresponding to the whole angiomotin molecule or fragments thereof, for generating immune responses which hinder the formation of blood vessels (angiogenesis). The invention provides vaccination with an angiomotin molecule and methods of using the vaccine to prevent formation of blood vessels that are critical for tumor growth, as well as other diseases produced or exacerbated by neoangiogenesis. We have shown that angiomotin vaccination provides anti-tumour protection.

### SUMMARY OF THE INVENTION

A first aspect of the invention provides the use of an angiomotin molecule or polynucleotide encoding an angiomotin molecule in the manufacture of a medicament (vaccine) for vaccinating a subject with or at risk of an angiogenesis-related disease or disorder.

A second aspect of the invention provides a method for treating a subject with or at risk of an angiogenesis-related disease or disorder, the method comprising the step of vaccinating the subject using a vaccine comprising an angiomotin molecule or polynucleotide encoding an angiomotin molecule.

The medicament or treatment may be a prophylactic treatment or a therapeutic treatment.

The vaccination may be performed by administering the angiomotin molecule or polynucleotide encoding an angiomotin molecule to the subject; or by exposing immune cells of the subject to the angiomotin molecule or polynucleotide encoding an angiomotin molecule outside the subject's body, followed by returning the exposed immune cells (and/or their progeny) to the subject.

The vaccine may comprise the complete protein (sequence 1) or portions thereof or the nucleotide sequence encoding the protein (sequence 2) or portions thereof. The scope of vaccinating with angiomotin as the immunogen is not restricted and encompasses protein, peptide and gene-based vaccination strategies. For example, the vaccine may comprise angiomotin or an immunostimulatory derivative or fragment of angiomotin. It may be desirable to administer both protein or peptide-based and polynucleotide/gene-based vaccine components to an individual, either at the same time or sequentially. This may promote a stronger, broader or more balanced immune response.

Derivatives include, but are not limited to, A) Analogues of angiomotin peptides, where the amino acid sequence of the native protein is modified at one or more amino acid positions, to increase the immunogenicity of the molecule. B) Chemical modification of one or more amino acids such as substitution of one or more chemical groups naturally occurring on the said amino acid with an artificial chemical group to increase the immunogenicity of the resultant peptide C) Conjugation of a peptide corresponding to a fragment of angiomotin with an immunogenic protein (e.g. keyhole limpet hemocyanin KLH) or a hapten (small chemical molecule such as dinitrophenol (DNP).

A further aspect of the invention provides a vaccine effective against blood vessel formation (for example angiogenesis), comprising an effective amount of the angiomotin molecule and/or polynucleotide as defined in relation to the preceding aspect of the invention. The vaccine is considered to be capable of generating an immune response against endogenous angiomotin in the recipient. For example a vaccine effective against blood vessel formation in a human may comprise an effective amount of a human angiomotin molecule and/or polynucleotide encoding a human angiomotin molecule. The angiomotin molecule or encoded angiomotin polypeptide may be full length angiomotin or a fragment thereof that promotes an immune response against epitope(s) that are present and accessible in endogenous angiomotin.

The vaccine may further comprise (as antigen(s)) one or more tumor antigen as well as other known angiogenic factor(s), for example an angiostatin receptor.

For example, as shown in the accompanying examples a plasmid encoding the transmembrane extracellular (TMEC) portion of the Her2/neu tumour antigen, a fragment of an oncogene, acts synergistically with a plasmid encoding a human angiomotin molecule so as to reduce tumour development in mice. Therefore, Her2/neu is an example of a tumour antigen which may comprise part of a vaccine of this aspect of the invention. In this example, TMEC is derived from the transmembrane and extracellular (TMEC) portion of the rat p185 (TMEC), which is the homologue of the human Her2/neu oncogene. This exemplifies how an angiomotin based vaccine may act in synergy as an “adjuvant” with a tumor antigen, which in this example is derived from Her2/neu but which could be derived from any tumor antigen expressed in human tumors, including but not restricted to examples such as those derived from the Cancer/testis tumor antigens (e.g. the MAGE, BAGE, GAGE, NY-ESO-1 family of antigens), the differentiation antigens (e.g. MART-1/MelanA, MC1R, Gp100, PSA, PSM, Tyrosinase, TRP-1 and -2), the broadly expressed antigens ART-4, CAMEL, CEA, Cyp-B, hTERT, iCE, MUC1 and 2, PRAME, P15, RUI and 2, SART-1 and 3, WT1) and other more unique or shared antigens (e.g. AFP, b-Catenin, Caspase-8, CDK-4, ELF2, G250, HSP70, HST-2, KIA A0205, MUM-1,2 and 3, RAGE) or viral antigens (e.g. HPV-E7, EBV antigens) or those derived from fusion proteins (e.g. those from bcr-abl, Del-cain, LDL/FUT, TEL/AML1). The tumor antigen could be administered as whole recombinant protein, or plasmid, or peptides, or fragments or parts of these (e.g. Class I or Class II epitopes).

The vaccine may also further comprise one or more antibodies against a tumor antigen or antigenic factor. Hence the invention also includes angiomotin vaccines combined with antibodies against tumor antigens, including but not restricted to antibodies to the Her2/neu antigen (e.g. Herceptin/Traztusumab) CD20 antigen on lymphomas, EpCAM antigen on colorectal cancer.

The vaccine is intended to generate an immune response against the angiomotin molecule. The resultant immune response can inhibit the formation of new capillaries that is required for the generation and/or sustenance of tumors and other disease states. Further (or alternatively), although angiomotin has not been detected in tumor cells hitherto, small amounts of angiomotin may be expressed by certain malignancies and may therefore serve as a tumor specific antigen. Whilst not being bound by theory, the present invention encompasses vaccination using an angiomotin vaccine (ie as described herein) where specific T cells and/or antibodies reactive to angiomotin recognize and destroy the tumor cells expressing angiomotin on its surface. All modes and approaches for vaccination using angiomotin as a tumor specific antigen use the same methods as indicated for vaccination for anti-angiogenic effects.

As will be well known to those skilled in the art, the choice of molecule and mode of administration for a vaccine (ie an agent acting through the recipient's immune system) differ from those for a direct therapeutic agent. For example, differences between an angiomotin molecule or polynucleotide encoding an angiomotin molecule administered as a vaccine, as opposed to a non-vaccine angiomotin therapeutic entity may include one or more of the following.

A non-vaccine therapeutic entity has to maintain a function of the angiomotin molecule (for example ability to bind angiostatin; or ability to interact with cellular components). The vaccine molecule on the other hand does not have to be a functioning angiomotin molecule. It can be (though does not have to be) the smallest non-functioning derivative (including fragment) or analogue that generates an immune response which is immunologically cross reactive with the native angiomotin molecule.

Usually a vaccine dose is one or two orders of magnitude lower than a non vaccine therapeutic dose, for example as measured on a “per kg bodyweight” basis.

Vaccines may include (or be accompanied by) “adjuvants” such as cytokines, BCG, alum etc that boost the immune response. There is no such accompaniment for non vaccine therapy. An adjuvant may be particularly important in the present case when, for example, immunizing against a “self” protein ie when immunizing against a human protein in a human.

Particularly considering the mechanism of action of the angiomotin molecule (in retarding angiogenesis) non-vaccine therapeutic entities would have to be administered almost daily, as opposed to vaccine administration that involves boosters once in two or three weeks. For example one or two administrations, at intervals of a few weeks, may be necessary, for either a gene vaccine or a polypeptide/peptide vaccine.

Non-vaccine vectors should have the capability of expressing the functioning molecules at high levels; this is very difficult to achieve in practice. DNA vaccines can express the immunizing antigen at much lower levels. Many suitable vectors and promoter systems are know, including the CMV promoter-based system used in the Examples. The immunizing antigen can be the minimum nonfunctional peptide or domain of angiomotin capable and sufficient of generating an immune response (antibody and/or T-cell) against native angiomotin. A functional angiomotin molecule need not be used.

In an embodiment of the present invention, a vaccine comprising or encoding a angiomotin or a fragment or derivative thereof is administered locally, topically, systemically or enterally to generate long lasting immunity against the molecule. The resulting immune response hinders the formation of new blood vessels. Prevention of neovascularization exerts a prophylactic or therapeutic effect on tumor formation or other vasculogenesis/angiogenesis-related disease.

In another embodiment of the invention, a patient's immune cells are stimulated ex vivo (outside the patient's body) by an angiomotin molecule (which may be, for example, a fragment of full-length angiomotin). This angiomotin molecule may be presented to the patient's immune cells, in particular T cells, following transfection of a polynucleotide encoding an angiomotin molecule (which may be, for example, a fragment of full-length angiomotin), into so called antigen presenting cells. The antigen presenting cell could also be pretreated externally with the angiomotin protein or with peptides derived from the angiomotin protein. Any cell type with the capacity to stimulate lymphocytes are here operationally defined as antigen presenting cell; these are considered to include so called Dendritic cells derived from monocytes or from lymphoid cells of the bone-marrow; and B cells, stimulated with mitogens or immortalized by s.c. Epstein Barr Virus.

Adoptive transfer of the stimulated immune cells back into the patient may result in the inhibition of neoangiogenesis through recognition of angiomotin expressed in the endothelial cells by the transferred immune cells, mainly T cells of CD8+ or CD4+ type and consequently restriction of the progress of tumors or other angiogenesis-related disease. Adoptive transfer of the stimulated immune cells back into the patient may also result in the restriction of the progress of tumors through recognition of angiomotin expressed in the tumor cells as a tumor antigen by the transferred immune cells, mainly T cells of CD8+ or CD4+ type.

Vasculogenesis/angiogenesis-related diseases include cancer (particularly solid tumours), hemangioma, ocular neovascularisation, diabetic retinopathy, macular degeneration, rheumatoid arthritis, inflammatory conditions (such as psoriasis, chronic inflammation of the intestines, asthma) and endometriosis.

A patient at risk of vasculogenesis/angiogenesis-related disease may be a patient at risk of cancer, particularly at risk of a solid tumour, for example a patient with a genetic predisposition to a form of cancer leading to a solid tumour, or a patient at lo environmental risk of a solid tumour. For example, a patient at risk of cancer may be a person with familial history of cancer who present polyps in the colon; or a woman known to be infected with a strain of human papilloma virus linked with cervical cancer and/or presenting pap smears demonstrating the earliest changes of cervical cancer.

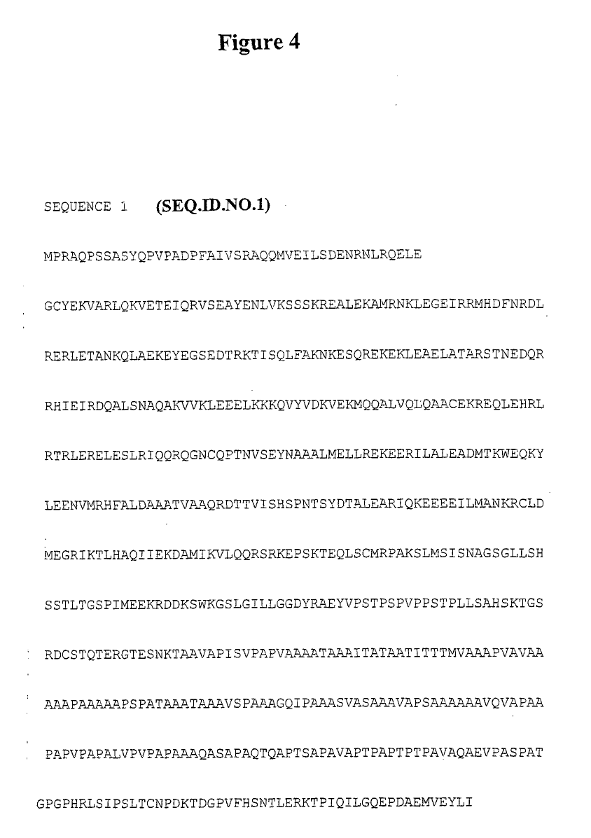

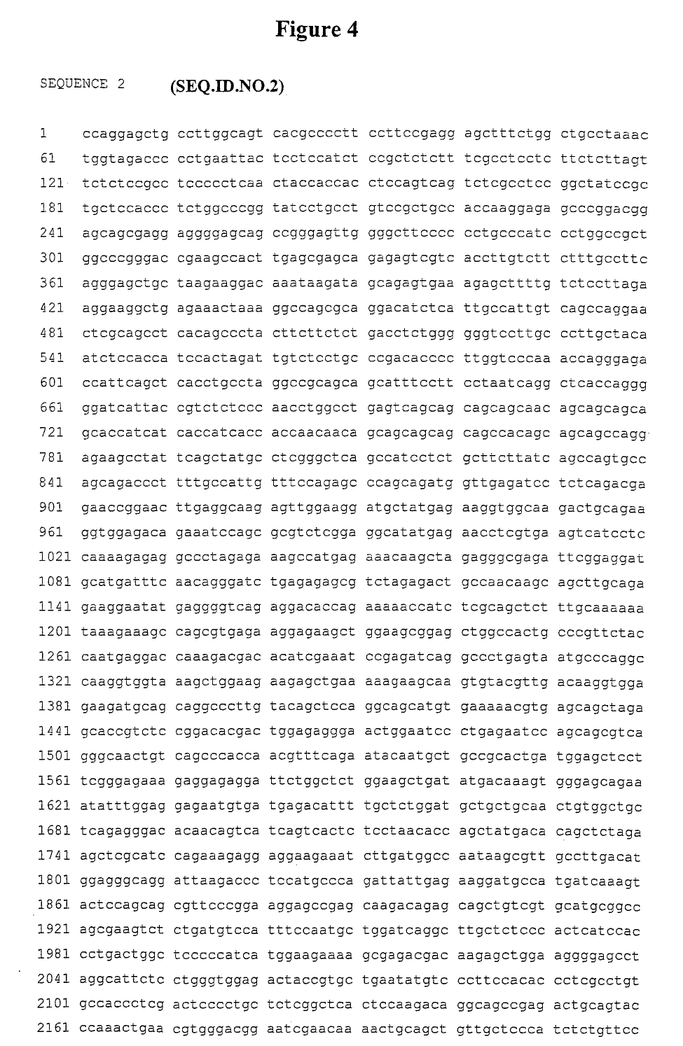

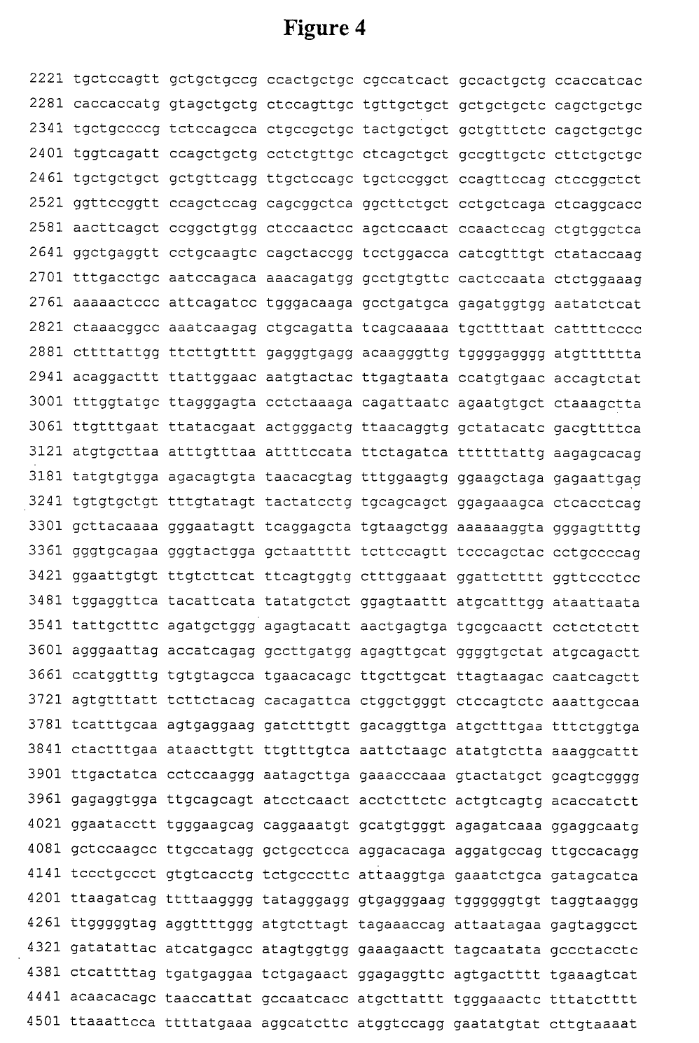

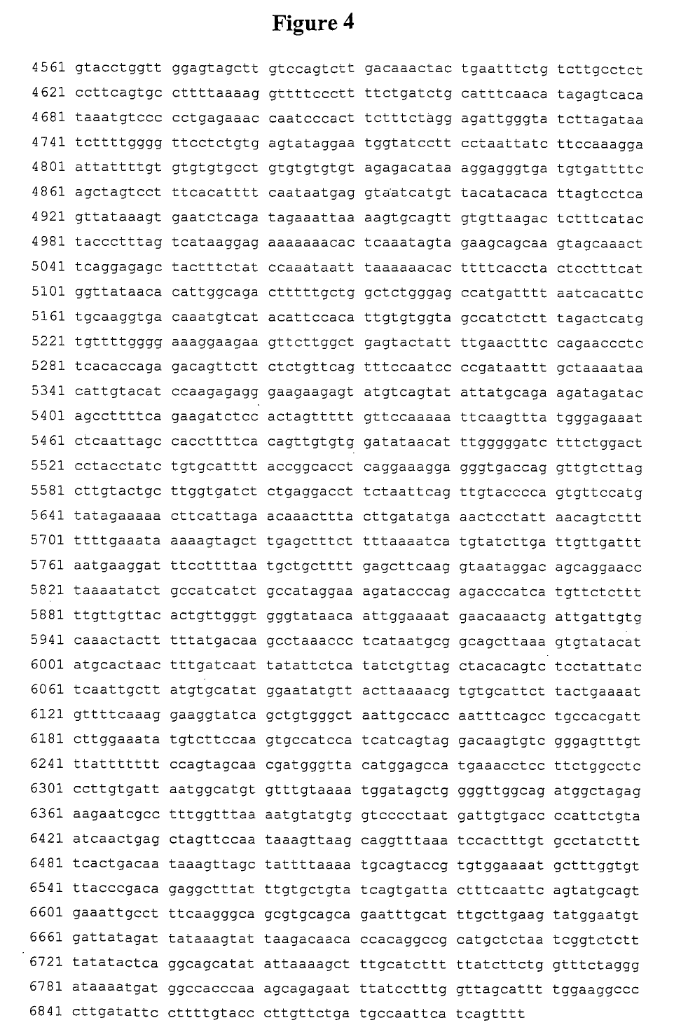

By “angiomotin molecule” is included any full length naturally occurring angiomotin polypeptide or fragment thereof, or any variant either thereof which retains antigenic cross-reactivity with the naturally occurring angiomotin polypeptide or fragment thereof. The term “angiomotin” is well known to those skilled in the art, and includes a polypeptide which has coiled-coil and C-terminal PDZ binding domains, is considered to be a cell surface-associated protein with an estimated molecular mass of 72 kDa. It is considered to bind to angiostatin and to mediate inhibitory effects of angiostatin on endothelial cell migration and tube formation. Examples of naturally occurring angiomotin polypeptides are given in the following: Troyanovsky et al (2001) J Cell Biol 152, 1247-1254; WO 99/66038; Levchenko et al (2003) J Cell Sci 116, 3803-3810; Bratt et al (2002) Gene 298(1), 69-77; GenBank accession No NP_{—}573572 (human). An angiomotin polypeptide may have at least 50%, 60% to 70%, 70% to 80%, 80 to 90% or 90 to 95% sequence identity with a naturally occurring angiomotin polypeptide sequence, for example as given in one of the listed references or accession numbers or in Sequence 1. In an embodiment, the angiomotin polypeptide has between 95% and 100% sequence identity with a naturally occurring angiomotin polypeptide sequence, for example the sequence of NP_{—}573572 or Sequence 1. The angiomotin polypeptide may be a “motin” family member as described in Bratt et al (2002) ie angiomotin-like1 (amotl1) or angiomotin-like 2 (amotl2). If a fragment of such a polypeptide is used then it is preferred that it is a fragment that encompasses a region of the motin polypeptide that has a corresponding region in the angiomotin polypeptide. Such regions are indicated in Bratt et al (2002), for example in FIG. 4, and may include part or all of the coiled coil domain region and/or the PDZ binding region. Suitable fragments of amot1 may include at least part of amino acids 439 to 956 of amot 1. Suitable fragments of amot2 may include at least part of amino acids 307 to 779 of amot 2.

References providing methods of assessing sequence identity are discussed further below.

The present invention also includes angiomotin derived polypeptides with or without glycosylation. Polypeptides expressed in yeast or mammalian expression systems may be similar to or slightly different in molecular weight and glycosylation pattern to the native molecules, depending upon the expression system. For instance, expression of DNA encoding polypeptides in bacteria such as E. coli typically provides non-glycosylated molecules. N-glycosylation sites of eukaryotic proteins are characterized by the amino acid triplet Asn-A.sub.1-Z, where A.sub.1 is any amino acid except Pro, and Z is Ser or Thr. Variants of polypeptides having inactivated N-glycosylation sites can be produced by techniques known to those of ordinary skill in the art, such as oligonucleotide synthesis and ligation or site-specific mutagenesis techniques, and are within the scope of this invention. Alternatively, N-linked glycosylation sites can be added to an angiomotin polypeptide.

More specifically, the disclosure of the present invention demonstrates that an angiomotin based vaccine can elicit a tumor rejection response, which may mean that a thymus-dependent lymphocyte (hereinafter “T cell”) response has been elicited. Therefore, the autochthoncus immune T cell response evoked by vaccination based on angiomotin can be utilized prophylactically or to treat malignancies or other vasculogenesis/angiogenesis related diseases. The present invention also provides, in another aspect, that nucleic acid molecules directing the expression of such a peptide may be used alone or in a viral vector for immunization.

Epitope sequences may be identified by techniques well known to those skilled in the art. For example, epitope mapping techniques such as those described in Epitope Mapping Protocols (1996) Methods Mol Biol 66, Glenn E Morris, Ed, Humana Press, Totowa, N.J.; U.S. Pat. No. 4,708,871; Geysen et al (1984) PNAS 81, 3998-4002; Geysen et al (1986) Molec Immunol 23, 709-715 may be used. Linear or conformational epitopes may be identified using such methods, for example using X-ray crystallography or 2D nuclear magnetic resonance-derived structural data. Antigenicity or hydrophobicity plots (such as generated using the OMIGA software available from Oxford Molecular Group, based on the algorithms of Hopp et al (1981) PNAS 78, 3824-3828 and Kyte et al (1982) J Mol Biol 157, 105-132) may also be useful in identifying epitopes.

Epitopes or compositions or whole cell vaccines expressing angiomotin or epitopes thereof as detailed below may be tested in, for example, the mouse model of tumour development described in the Examples in order to confirm the generation of an immune response and/or an effect on angiogenesis and tumour development. Mouse HLA-A2 transgenic mouse models, or mouse models transgenic for other human HLA class I or II antigens, may be used. Other appropriate models for testing the effect of angiomotin based vaccines include transgenic mouse models which spontaneously develop tumors, such as the neu-T BALB/c model which develop breast carcinomas. The ability of angiomotin based vaccination to activate the host immune system in these models may be assessed by using known immunological assays measuring CD8+ and CD4+ T cell responses, such as ELISPOT assays, cytokine release assays and T cell proliferation assays as well as assays to measure antibody responses, such as ELISA assays and Flow cytometry based assays. To determine whether angiogenesis is inhibited in mice immunized with angiomotin or other compositions based on angiomotin mentioned herein, the development of neovascularization in the tumor may be assessed by various known assays, including the “Matrigel-plug” assay (as well known to those skilled in the art and as described in (for example) London et al (2003) Cancer Gene Ther 10(11), 823-832) or imaging based assays such as the skin-flap window-chamber model.

The recipient may be human, for example a human with or at risk of an angiogenesis-associated disease or condition, for example a solid tumour, hemangioma, endometriosis, ocular neovascularisation, diabetic retinopathy, macular degeneration, rheumatoid arthritis, inflammatory conditions (such as psoriasis, chronic inflammation of the intestines, asthma). Alternatively, the recipient may be an animal with or at risk of such a condition, for example a domesticated animal (eg a companion animal such as a cat or dog) or animal important in agriculture (ie livestock), for example cattle, sheep, goats, or poultry, for example chickens and turkeys.

In an embodiment the polypeptide or polypeptides comprise at least a domain of angiomotin ie a portion of angiomotin that is capable of, or predicted to be capable of, folding independently in a manner similar to that in which it would fold in full length angiomotin. Methods of identifying such domains using computer analysis (for example incorporating analysis of hydrophobicity and/or likelihood to form an α helix or strand of a β sheet) are well known to those skilled in the art. It is considered that the ability to fold independently may be important in generating an antibody response to native angiomotin. However, it is considered that folding does not matter when it comes to inducing an immune response based on T cells, as angiomotin will be degraded by antigen presenting cells into small peptide fragments and presented to the T cells as such; folding or conformation is not considered to matter here, only amino acid sequence. It is considered that the angiomotin vaccine acts principally by inducing an immune response based on T cells and that it may not be necessary to use a domain which is capable of independent folding.

In an embodiment the polypeptide or polypeptides comprise full length angiomotin.

One or more peptides or peptidomimetic compounds representing one or more epitope(s) of angiomotin (for example as discussed in Example 1) may be used. For example, short peptides, for example of up to about 15, 12, 10 or 9 amino acids (or polynucleotides encoding such short peptides) may be used. By epitopes is included mimotopes, as well known to those skilled in the art.

An antibody may be used as an antigen in a vaccine. For example, an antibody to an antibody to the intended target (eg angiomotin) is administered, then the B cells of the immune system make antibodies to that antibody that also recognize the endogenous angiomotin. This is called an anti-idiotype vaccine, and is different from passive antibody therapy, in which an antibody to the intended target is administered. Such an anti-idiotypic antibody is included within the definition of an “angiomotin molecule” as used herein.

The invention also provides a combination between an angiomotin based vaccine and other types of antiangiogenic therapies targeting endothelial cells or products. Thus, for example, the vaccine may further comprise (in addition to the angiomotin molecule or polypeptide) a polynucleotide or polypeptide (or peptidomimetic compound) suitable for acting as an immunogen against an angiogenesis-promoting polypeptide (other than angiomotin), for example VEGFR-2 (for example Accession No AF063658) or Tie2 (for example Accession No BC035514). Thus, for example, the invention includes (in combination with anti-angiomotin vaccination) vaccination with vascular endothelial growth factor receptor 2 (VEGFR2), for example administered as pDNA vaccine, viral vector, or expressed in DC cells or loaded onto DC cells. There are many other types of anti-angiogenic therapies that may be useful in combination with anti-angiomotin vaccination. Preferences for angiogenesis-promoting polypeptide sequences correspond to the preferences (suitably adapted) as set out for angiomotin. These other angiogenesis-promoting polypeptide-derived sequences (for example VEGFR-2 sequences) may be included in the same or a different polypeptide (or polynucleotide, as appropriate) as the angiomotin-derived sequences. The treatment or vaccine may further comprise a combination between an angiomotin based vaccine and immunotherapy based on administration of cytokines with anti-tumor effects or of tumor antigens, for example administered as pDNA vaccine, viral vector, or expressed in DC cells or loaded onto DC cells.

By angiomotin is included variants, fragments and fusions that have antigenicity (for example as assessed by anti-angiogenic effect), interactions or activities which are substantially the same as those of the angiomotin sequences described herein and/or those disclosed in references (including public database references) cited above and/or other public database records. It is preferred that the angiomotin or fragment thereof is a naturally occurring angiomotin or fragment thereof, or a fusion of such an angiomotin or fragment with a non-angiomotin-derived polypeptide. For example, the angiomotin-derived sequence may be fused with a moiety that aids expression, stability and/or purification, for example a maltose binding protein (MBP) moiety or His tag, as well known to those skilled in the art.

A “variant” will have a region which has at least 50% (preferably 60, 70, 80, 90, 95 or 99%) sequence identity with an angiomotin polypeptide as described herein or in the references indicated above, as measured by the Bestfit Program of the Wisconsin Sequence Analysis Package, version 8 for Unix. The percentage identity may be calculated by reference to a region of at least 50 amino acids (preferably at least 60, 75, or 100) of the candidate variant molecule, and the most similar region of equivalent length in the angiomotin sequence, allowing gaps of up to 5%.

The percent identity may be determined, for example, by comparing sequence information using the GAP computer program, version 6.0 described by Devereux et al. (Nucl. Acids res. 12:387, 1984) and available from the University of Wisconsin Genetics Computer Group (UWGCG). The GAP program utilizes the alignment method of Neddleman and Wunsch (J. Mol. Biol. 48:443, 1970), as revised by Smith and Waterman (Adv. Appl. Math 2.482. 1981). The preferred default parameters for the GAP program include: (1) a comparison matrix (containing a value of 1 for identities and 0 for non-identities) for nucleotides, and the weighted comparison matrix of Bribskov and Burgess, Nucl. Acids Res. 14:6745, 1986 as described by Schwarts and Dayhoff, eds, Atlas of Protein Sequence and Structure, National Biomedical Research Foundation, pp 353-358, 1979; (2) a penalty of 3.0 for each gap and an additional 0.10 penalty for each symbol in each gap; and (3) no penalty for end gaps.

Preferably, the angiomotin polypeptide consists of a variant, or fusion (or a fragment) of a given full length wild-type angiomotin which is antigenically cross-reactive with the said native full length wild-type angiomotin.

Preferred angiomotin sequences are shown in, for example Sequence 1 and 2:

Substitutions, deletions, insertions or any subcombination may be used to arrive at a final construct. Since there are 64 possible codon sequences but only twenty known amino acids, the genetic code is degenerate in the sense that different codons may yield the same amino acid. Thus there is at least one codon for each amino acid, ie each codon yields a single amino acid and no other. It will be apparent that during translation, the proper reading frame must be maintained in order to obtain the proper amino acid sequence in the polypeptide ultimately produced.

Techniques for additions, deletions or substitutions at predetermined amino acid sites having a known sequence are well known. Exemplary techniques include oligonucleotide-mediated site-directed mutagenesis and the polymerase chain reaction.

Oligonucleotide site-directed mutagenesis in essence involves hybridizing an oligonucleotide coding for a desired mutation with a single strand of DNA containing the region to be mutated and using the single strand as a template for extension of the oligonucleotide to produce a strand containing the mutation. This technique, in various forms, is described in Zoller and Smith (1982) Nucl. Acids Res. 10, 6487.

The vaccine antigen may comprise more than one angiomotin-derived epitope. An epitope is defined as a polypeptide recognized by CD4+ or CD8+ T cells. This may be useful in promoting an immune response, as well known to those skilled in the art.

The vaccine may comprise further polypeptides or polynucleotides, as will be apparent to those skilled in the art. The polypeptide(s) or polynucleotide(s) may, for example, be included in the vaccine in the form of a recombinant organism or part thereof, or product (such as a cell culture supernatant) thereof, preferably microorganism, preferably capable of expressing the polypeptides(s) ie capable of expressing the angiomotin amino acid sequences, or alternatively capable of delivering nucleic acid encoding the polypeptide(s) to a host cell for expression therein. The recombinant microorganism is preferably a non-virulent microorganism, as well known to those skilled in the art. The recombinant microorganism may be, for example, a Bifidobacterium or a lactobacillus, or an attenuated Salmonella or BCG or attenuated E. coli. The recombinant organism may alternatively be a plant, for example making use of the teaching of WO97/40177.

In a further alternative, the vaccine can be made either of whole eukaryotic cells or of substances contained by the cells. For example the cells may be derived from the type of organism for which the vaccine is intended. For example the cells may be human cells. The cells may be recombinant cells. The cells may be cells of a cell line capable of expressing an angiomotin molecule, for example transfected with a polynucleotide encoding an angiomotin molecule. Thus, the cells may comprise a recombinant polynucleotide encoding an angiomotin molecule, and express a recombinant angiomotin molecule. The cells may be irradiated, heat killed or paraformaldehyde-fixed and used for immunization.

The cells may be tumour cells which express angiomotin, or freshly explanted or cultured endothelial cells, of human origin or xenogeneic from another species, expressing angiomotin or cells which are transfected with and express angiomotin or parts of this molecule. The cells may be tested to determine that they express angiomotin. The cells may be antigen presenting cells, such as dendritic cells (DC), which are loaded with the angiomotin molecule or transfected with cDNA or mRNA that encode angiomotin or angiomotin associated products. A tumour cell vaccine (which may not necessarily comprise an angiomotin antigen) may be used alongside the angiomotin vaccine. For a whole cell vaccine, tumor cells are taken out of the patient(s), and grown in the laboratory. Then the tumor cells are treated to make sure that 1) they can no longer multiply, and 2) there is nothing present that could infect the patient. When whole tumor cells are injected into a person, an immune response against the antigens on the tumor cells is generated.

There are two types of whole cell cancer vaccines. An autologous whole cell vaccine is made with the patient's own whole, inactivated tumor cells. An allogenic whole cell vaccine is made with someone else's whole, inactivated tumor cells or several peoples' tumor cells combined.

APC vaccines are made of the cells that are best at turning on T cells to kill tumor cells, the antigen presenting cells (APCs). The most common type of APC used is the dendritic cell. Cancer vaccines, for example, can be made of dendritic cells that have been primed, or grown in the presence of, tumor antigens in the laboratory. Dendritic cells (or APCs) primed with antigen carry the tumor antigens on their surface and when injected, are ready to strongly activate T cells to multiply and to kill tumor cells. The same approach can be used with an angiomotin molecule as the antigen.

Antigen vaccines are not made of whole cells, but of one or more antigens contained by the tumor. One tumor can have many antigens. Some antigens are common to all cancers of a particular type, and some antigens are unique to an individual. A few antigens are shared between tumors of different types of cancer. There are many ways to deliver the antigens in an antigen vaccine. Proteins or pieces of protein from the tumor cells can be given directly as the vaccine.

The angiomotin molecule (or other vaccine antigen) may be a peptidomimetic compound, for example corresponding to an angiomotin epitope or mimotope as discussed above. The term “peptidomimetic” refers to a compound that mimics the conformation and desirable features of a particular peptide as a therapeutic agent, but that avoids the undesirable features. For example, morphine is a compound which can be orally administered, and which is a peptidomimetic of the peptide endorphin.

Therapeutic applications involving peptides are limited, due to lack of oral bioavailability and to proteolytic degradation. Typically, for example, peptides are rapidly degraded in vivo by exo- and endopeptidases, resulting in generally very short biological half-lives. Another deficiency of peptides as potential therapeutic agents is their lack of bioavailability via oral administration. Degradation of the peptides by proteolytic enzymes in the gastrointestinal tract is likely to be an important contributing factor. The problem is, however, more complicated because it has been recognised that even small, cyclic peptides which are not subject to rapid metabolite inactivation nevertheless exhibit poor oral bioavailability. This is likely to be due to poor transport across the intestinal membrane and rapid clearance from the blood by hepatic extraction and subsequent excretion into the intestine. These observations suggest that multiple amide bonds may interfere with oral bioavailability. It is thought that the peptide bonds linking the amino acid residues in the peptide chain may break apart when the peptide drug is orally administered.

There are a number of different approaches to the design and synthesis of peptidomimetics. In one approach, such as disclosed by Sherman and Spatola, J. Am. Chem. Soc., 112: 433 (1990), one or more amide bonds have been replaced in an essentially isoteric manner by a variety of chemical functional groups. This stepwise approach has met with some success in that active analogues have been obtained. In some instances, these analogues have been shown to possess longer biological half-lives than their naturally-occurring counterparts. Nevertheless, this approach has limitations. Successful replacement of more than one amide bond has been rare. Consequently, the resulting analogues have remained susceptible to enzymatic inactivation elsewhere in the molecule. When replacing the peptide bond it is preferred that the new linker moiety has substantially the same charge distribution and substantially the same planarity as a peptide bond.

Retro-inverso peptidomimetics, in which the peptide bonds are reversed, can be synthesised by methods known in the art, for example such as those described in Mézière et al (1997) J. Immunol. 159 3230-3237. This approach involves making pseudopeptides containing changes involving the backbone, and not the orientation of side chains. Retro-inverse peptides, which contain NH—CO bonds instead of CO—NH peptide bonds, are much more resistant to proteolysis.

In another approach, a variety of uncoded or modified amino acids such as D-amino acids and N-methyl amino acids have been used to modify mammalian peptides. Alternatively, a presumed bioactive conformation has been stabilised by a covalent modification, such as cyclisation or by incorporation of γ-lactam or other types of bridges. See, eg. Veber et al, Proc. Natl. Acad. Sci. USA, 75:2636 (1978) and Thursell et al, Biochem. Biophys. Res. Comm., 111:166 (1983).

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

One approach to the synthesis of cyclic stabilised peptidomimetics is ring closing metathesis (RCM). This method involves steps of synthesising a peptide precursor and contacting it with a RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Polypeptides in which one or more of the amino acid residues are chemically modified, before or after the polypeptide is synthesised, may be used as antigen providing that the function of the polypeptide, namely the production of a specific immune response in vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the polypeptide from in vivo metabolism. The polypeptide may be mannosylated or otherwise modified to increase its antigenicity, or combined with a compound for increasing its antigenicity and/or immunogenicity.

The use of agonistic epitopes derived from angiomotin is also included in the present invention. Agonistic epitopes are designed to more efficiently activate the immune system through a more effective activation of MHC class I or MHC class II restricted CD8 or CD4+ T cells. Two general approaches are included in the invention to design agonist epitopes from T cell epitopes. One approach entails modification of HLA anchor residues, resulting in higher HLA class I or class II binding. This approach has been applied with success for several HLA class I binding peptides derived from tumor antigens or microbial antigens. Alternatively, the replacement of residues involved in the T cell receptor (abbreviated TCR) contact may also result in an increased response by T cells and is intended to be covered by this invention.

Generally, as well known to those skilled in the art, and as described in, for example, U.S. Pat. No. 5,869,445, amino acid substitutions may be made in a variety of ways to provide other embodiments of variants within the present invention. First, for example, amino acid substitutions may be made conservatively; i.e., a substitute amino acid replaces an amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. An example of a non-conservative change is to replace an amino acid of one group with an amino acid from another group.

Another way to make amino acid substitutions to produce variants of the present invention is to identify and replace amino acids in T cell motifs with potential to bind to class II MHC molecules (for CD4+ T cell response) or class I MHC molecules (for CD8+ T cell response). Peptide segments (of an angiomotin molecule) with a motif with theoretical potential to bind to class II MHC molecules may be identified by computer analysis. For example, a protein sequence analysis package, T Sites, that incorporates several computer algorithms designed to distinguish potential sites for T cell recognition can be used (Feller and de la Cruz, Nature 349:720-721, 1991). Two searching algorithms are used: (1) the AMPHI algorithm described by Margalit (Feller and de la Cruz, Nature 349:720-721, 1991; Margalit et al., J. Immunol. 138:2213-2229, 1987) identifies epitope motifs according to alpha-helical periodicity and amphipathicity; (2) the Rothbard and Taylor algorithm identifies epitope motifs according to charge and polarity pattern (Rothbard and Taylor, EMBO 7:93-100, 1988). Segments with both motifs are most appropriate for binding to class II MHC molecules. CD8+ T cells recognize peptide bound to class I MHC molecules. Falk et al. have determined that peptides binding to particular MHC molecules share discernible sequence motifs (Falk et al., Nature 351:290-296, 1991). A peptide motif for binding in the groove of HLA-A2.1 has been defined by Edman degradation of peptides stripped from HLA-A2.1 molecules of a cultured cell line (Table 2, from Falk et al., supra). The method identified the typical or average HLA-A2.1 binding peptide as being 9 amino acids in length with dominant anchor residues occurring at positions 2 (L) and 9 (V). Commonly occurring strong binding residues have been identified at positions 2 (M), 4 (E,K), 6 (V), and 8 (K). The identified motif represents the average of many binding peptides.

The epitope(s) (for example epitope-forming amino acid sequences, or regions considered to comprise anti-angiogenic epitopes) may be present as single copies or as multiples, for example tandem repeats. Such tandem or multiple repeats may be sufficiently antigenic themselves to obviate the use of a carrier. It may be advantageous for the polypeptide to be formed as a loop, with the N-terminal and C-terminal ends joined together, or to add one or more Cys residues to an end to increase antigenicity and/or to allow disulphide bonds to be formed. If the epitope, for example epitope-forming amino acid sequence, is covalently linked to a carrier, preferably a polypeptide, then the arrangement is preferably such that the epitope-forming amino acid sequence forms a loop.

According to current immunological theories, a carrier function should be present in any immunogenic formulation in order to stimulate, or enhance stimulation of, the immune system. The epitope(s) as defined above in relation to the preceding aspects of the invention may be associated, for example by cross-linking, with a separate carrier, such as serum albumins, myoglobins, bacterial toxoids and keyhole limpet haemocyanin. Angiomotin may itself act as a carrier or adjuvant. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both.

Alternatively, several copies of the same or different epitope may be cross-linked to one another; in this situation there is no separate carrier as such, but a carrier function may be provided by such cross-linking. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present). Any of the conventional ways of cross-linking polypeptides may be used, such as those generally described in O'Sullivan et al Anal. Biochem. (1979) 100, 100-108. For example, the first portion may be enriched with thiol groups and the second portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

Further useful cross-linking agents include S-acetylthioglycolic acid N-hydroxysuccinimide ester (SATA) which is a thiolating reagent for primary amines which allows deprotection of the sulphydryl group under mild conditions (Julian et al (1983) Anal. Biochem. 132, 68), dimethylsuberimidate dihydrochloride and N,N′-o-phenylenedimaleimide.

If the polypeptide is prepared by expression of a suitable nucleotide sequence in a suitable host, then it may be advantageous to express the polypeptide as a fusion product with a peptide sequence which acts as a carrier. Kabigen's “Ecosec” system is an example of such an arrangement.

Other adjuvants that may be useful include adjuvants discussed in WO 02/053181, for example VSA3, which includes DDA (see U.S. Pat. No. 5,951,988).

Suitable vectors or constructs which may be used to prepare a suitable recombinant polypeptide or polynucleotide will be known to those skilled in the art. A polynucleotide capable of expressing the required polypeptide or polypeptides may be prepared using techniques well known to those skilled in the art.

It may be desirable for the polynucleotide to be capable of expressing the polypeptide(s) in the recipient, so that the human or animal may be administered the polynucleotide, leading to expression of the antigenic polypeptides (ie sequences derived from angiomotin and optionally other polypeptides) in the human or animal. The polypeptide(s), for example angiomotin, may be expressed from any suitable polynucleotide (genetic construct) as is described below and delivered to the recipient. Typically, the genetic construct which expresses the polypeptide comprises the said polypeptide coding sequence operatively linked to a promoter which can express the transcribed polynucleotide (eg mRNA) molecule in a cell of the recipient, which may be translated to synthesise the said polypeptide. Suitable promoters will be known to those skilled in the art, and may include promoters for ubiquitously expressed genes, for example housekeeping genes or for tissue-selective genes, depending upon where it is desired to express the said polypeptide (for example, in dendritic cells or other antigen presenting cells or precursors thereof). Preferably, a dendritic cell or dendritic precursor cell-selective promoter is used, but this is not essential, particularly if delivery or uptake of the polynucleotide is targeted to the selected cells, eg dendritic cells or precursors. Dendritic cell-selective promoters may include the CD83 or CD36 promoters.

Other polypeptides/proteins which may desirably be expressed or combined with the angiomotin based vaccine include immunostimulatory agents such as cytokines or growth factors. Examples include GM-CSF, IL-2, IL12 or IL-15 (see, for example Example 1). Other immunostimulatory agents which may be expressed or included are factors that bind to so called Toll receptors, which include the small immunostimulatory molecule Imiquimod, microbial products such as flagellin or unmethylated bacterial CpG motifs or oligonucleotides based on CpG motifs.

The nucleic acid sequence capable of expressing the polypeptide(s) is preferably operatively linked to regulatory elements necessary for expression of said sequence.

“Operatively linked” refers to juxtaposition such that the normal function of the components can be performed. Thus, a coding sequence “operatively linked” to regulatory elements refers to a configuration wherein the nucleic acid sequence encoding the antigen (or immunostimulatory molecule) can be expressed under the control of the regulatory sequences.

“Regulatory sequences” refers to nucleic acid sequences necessary for the expression of an operatively linked coding sequence in a particular host organism. For example, the regulatory sequences which are suitable for eukaryotic cells are promotors, polyadenylation signals, and enhancers.

“Vectors” means a DNA molecule comprising a single strand, double strand, circular or supercoiled DNA. Suitable vectors include retroviruses, adenoviruses, adeno-associated viruses, pox viruses and bacterial plasmids. Retroviral vectors are retroviruses that replicate by randomly integrating their genome into that of the host. Suitable retroviral vectors are described in WO 92/07573.

Viral vectors are intended not to make people sick or to carry any diseases. These viruses can be engineered in the laboratory so that when they infect a human cell, the cell will make and display the required antigen on its surface. The virus is capable of infecting only a small number of human cells—enough to start an immune response, but not enough to make a person sick.

Viruses can also be engineered to make cytokines or display proteins on their surface that help activate immune cells. These can be given alone or with a vaccine to help the immune response.

Adenovirus is a linear double-standard DNA Virus. Suitable adenoviral vectors are described in Rosenfeld et al, Science, 1991, Vol. 252, page 432.

Adeno-associated viruses (AAV) belong to the parvo virus family and consist of a single strand DNA of about 4-6 KB.

Pox viral vectors are large viruses and have several sites in which genes can be inserted. They are thermostable and can be stored at room temperature. Safety studies indicate that pox viral vectors are replication-defective and cannot be transmitted from host to host or to the environment.

Targeting the vaccine to specific cell populations, for example antigen presenting cells, may be achieved, for example, either by the site of injection, use of targeting vectors and delivery systems, or selective purification of such a cell population from the recipient and ex vivo administration of the peptide or nucleic acid (for example dendritic cells may be sorted as described in Zhou et al (1995) Blood 86, 3295-3301; Roth et al (1996) Scand. J. Immunology 43, 646-651). In addition, targeting vectors may comprise a tissue- or tumour-selective promoter which directs expression of the antigen at a suitable place.

Although the genetic construct can be DNA or RNA it is preferred if it is DNA.

Preferably, the genetic construct is adapted for delivery to a human cell.

Means and methods of introducing a genetic construct into a cell in or removed from an animal body are known in the art. For example, the constructs of the invention may be introduced into the cells by any convenient method, for example methods involving retroviruses, so that the construct is inserted into the genome of the (dividing) cell. Targeted retroviruses are available for use in the invention; for example, sequences conferring specific binding affinities may be engineered into pre-existing viral env genes (see Miller & Vile (1995) Faseb J. 9, 190-199 for a review of this and other targeted vectors for gene therapy).

Preferred retroviral vectors may be lentiviral vectors such as those described in Verma & Somia (1997) Nature 389, 239-242.

Other methods involve simple delivery of the construct into the cell for expression therein either for a limited time or, following integration into the genome, for a longer time. An example of the latter approach includes liposomes (Nässander et al (1992) Cancer Res. 52, 646-653). Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

Bacterial delivery methods which may be suitable are described in Dietrich (2000) Antisense Nucleic Acid Drug Delivery 10, 391-399. For example, attenuated bacterial strains allow the administration of recombinant vaccines via the mucosal surfaces. Whereas attenuated bacteria are generally engineered to express heterologous antigens, a further approach employs intracellular bacteria for the delivery of eukaryotic antigen expression vectors (DNA vaccines). This strategy allows a direct delivery of DNA to professional antigen-presenting cells (APC), such as macrophages and dendritic cells (DC), through bacterial infection. The bacteria used for DNA vaccine delivery either enter the host cell cytosol after phagocytosis by the APC, for example, Shigella and Listeria, or they remain in the phagosomal compartment, such as Salmonella. Both intracellular localizations of the bacterial carriers may be suitable for successful delivery of DNA vaccine vectors of the present invention.

Expression of the angiomotin (or other) polypeptide may be under the control of inducible bacterial promoters, for example promoters that are induced when the bacterium encounters or enters a host organism environment (for example the host's gut) or binds to or enters a host cell.

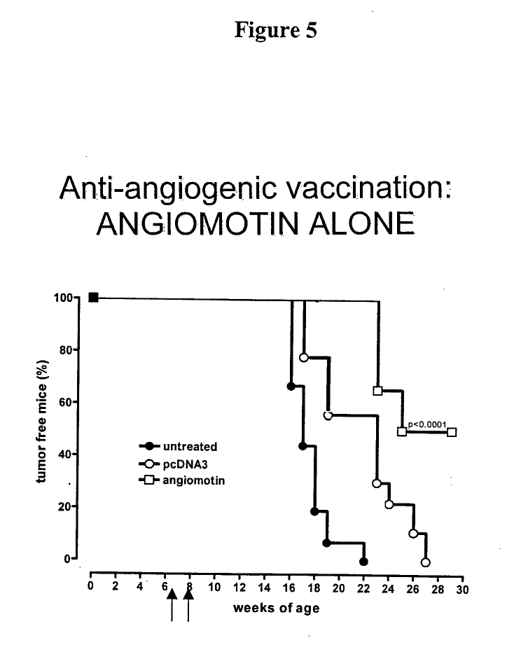

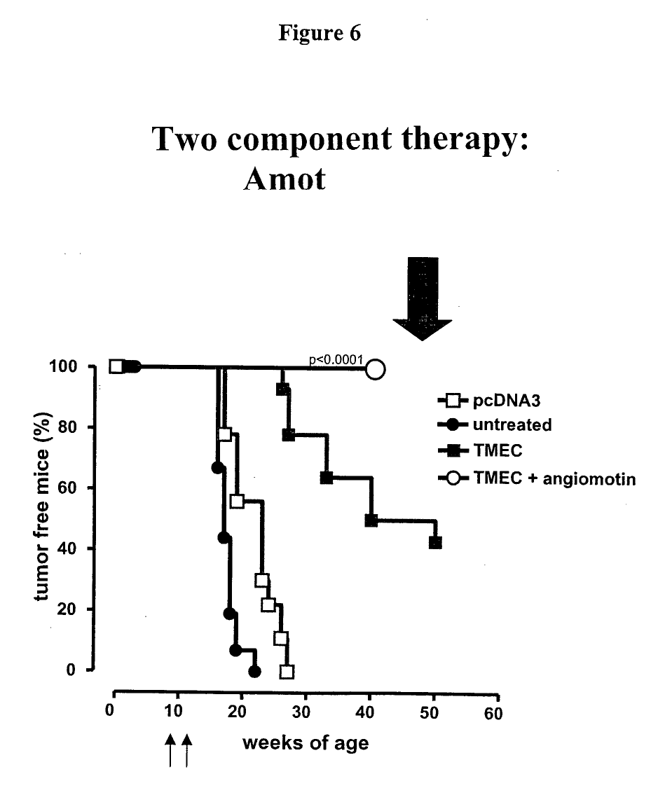

Gene gun delivery is a preferred method of delivery in relation to the present invention. In particular, the angiomotin plasmid DNA can be administered as “gene-gun” intradermal vaccination, intramuscular injection, or plasmid DNA vaccination injected intradermally or intramuscularly and then followed by cutaneous or intramuscular “electroporation” at the site of injection, to increase the efficacy of the vaccination. The use of a gene-gun to deliver a vaccine of the invention is described in example 5 and the data generated is shown in FIGS. 5 and 6.

The DNA may also be delivered by adenovirus wherein it is present within the adenovirus particle, for example, as described below.

A high-efficiency nucleic acid delivery system that uses receptor-mediated endocytosis to carry DNA macromolecules into cells may be employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the target cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

“Naked DNA” and DNA complexed with cationic and neutral lipids may also be useful in introducing the DNA of the invention into cells of the recipient. Non-viral approaches to gene therapy are described in Ledley (1995) Human Gene Therapy 6, 1129-1144. Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Other suitable viruses or virus-like particles include HSV, AAV, vaccinia, lentivirus and parvovirus.

Immunoliposomes (antibody-directed liposomes) are especially useful in targeting to cell types which over-express a cell surface protein for which antibodies are available, as is possible with dendritic cells or precursors, for example using antibodies to CD1, CD14 or CD83 (or other dendritic cell or precursor cell surface molecule, as indicated above). For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected, for example intraperitoneally or directly into a site where the target cells are present, for example subcutaneously.

It will be appreciated that it may be desirable to be able to regulate temporally expression of the polypeptide(s) (for example antigenic polypeptides) in the cell. Thus, it may be desirable that expression of the polypeptide(s) is directly or indirectly (see below) under the control of a promoter that may be regulated, for example by the concentration of a small molecule that may be administered to the recipient when it is desired to activate or repress (depending upon whether the small molecule effects activation or repression of the said promoter) expression of the polypeptide. It will be appreciated that this may be of particular benefit if the expression construct is stable ie capable of expressing the polypeptide (in the presence of any necessary regulatory molecules) in the said cell for a period of at least one week, one, two, three, four, five, six, eight months or one or more years. It is preferred that the expression construct is capable of expressing the polypeptide in the said cell for a period of less than one month. A preferred construct of the invention may comprise a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (reviews induction factors e.g., hormones, growth factors, cytokines, cytostatics, irradiation, heat shock and associated responsive elements). Tetracycline-inducible vectors may also be used. These are activated by a relatively-non toxic antibiotic that has been shown to be useful for regulating expression in mammalian cell cultures. Also, steroid-based inducers may be useful especially since the steroid receptor complex enters the nucleus where the DNA vector must be segregated prior to transcription.

This system may be further improved by regulating the expression at two levels, for example by using a tissue-selective promoter and a promoter controlled by an exogenous inducer/repressor, for example a small molecule inducer, as discussed above and known to those skilled in the art. Thus, one level of regulation may involve linking the appropriate polypeptide-encoding gene to an inducible promoter whilst a further level of regulation entails using a tissue-selective promoter to drive the gene encoding the requisite inducible transcription factor (which controls expression of the polypeptide (for example the antigenic polypeptide)-encoding gene from the inducible promoter). Control may further be improved by cell-type-specific targeting of the genetic construct.

The genetic constructs of the invention can be prepared using methods well known in the art.

The therapeutic agent or molecule (vaccine), for example antigenic molecule, for example a angiomotin molecule or construct encoding an angiomotin molecule or a formulation thereof, may be administered by any conventional method including oral and parenteral (eg subcutaneous or intramuscular) injection. Preferred routes include oral, intranasal or intramuscular injection. The treatment may consist of a single dose or a plurality of doses over a period of time. It will be appreciated that an inducer, for example small molecule inducer as discussed above may preferably be administered orally.

Methods of delivering genetic constructs, for example adenoviral vector constructs to cells of a recipient will be well known to those skilled in the art. In particular, an adoptive therapy protocol may be used or, more preferably, a gene gun may be used to deliver the construct to dendritic cells, for example in the skin.

Adoptive therapy protocols are described in Nestle et al (1998) Nature Med. 4, 328-332 and De Bruijn et al (1998) Cancer Res. 58, 724-731.

The therapeutic agent (vaccine) may be given to a subject who is being treated for the disease by some other method. Thus, although the method of treatment may be used alone it is desirable to use it as an adjuvant therapy, for example alongside conventional preventative or therapeutic methods or immunotherapy targeting tumour antigens. For example, combinations of the angiomotin vaccine with vaccines based on tumor antigens, administered as peptides, proteins, oligonucleotides or whole tumor cells may be suitable combination therapies. An example of a suitable tumor antigen is provided in the accompanying example and further examples are mentioned above in relation to the vaccine according to the invention.

Whilst it is possible for a therapeutic molecule as described herein, for example an antigenic molecule or immunostimulatory molecule, to be administered alone, it is preferable to present it as a pharmaceutical formulation, together with one or more acceptable carriers. The carrier(s) must be “acceptable” in the sense of being compatible with the therapeutic molecule (which may be a nucleic acid or polypeptide) and not deleterious to the recipients thereof. Typically, the carriers will be water or saline which will be sterile and pyrogen free.

The pharmaceutical composition may further comprise a component for increasing the antigenicity and/or immungenicity of the composition, for example an adjuvant and/or a cytokine, as discussed above. A polyvalent antigen (cluster of antigens) may be useful.

Commercial versions of cytokines have been used both as nonspecific immunotherapies to generally boost the immune system and as adjuvants given along with other immunotherapies such as tumor vaccines. GM-CSF is being tested against cancer as a nonspecific immunotherapy and as an adjuvant given with other types of immunotherapies.

A variety of other compounds are known to boost the activity of the immune system and are now under study as possible adjuvants, particularly for vaccine therapies. Some of the most commonly studied adjuvants are listed below, but many more are under development.

Levamisole, a drug first used against parasitic infections, has recently been found to improve survival rates among people with colorectal cancer when used together with some chemotherapy drugs. It is often used as an immunotherapy adjuvant because it can activate T lymphocytes. Levamisole is now used routinely for people with some stages of colorectal cancer and is being tested in clinical trials as a treatment for other types of cancer.

Aluminum hydroxide (alum) is one of the most common adjuvants used in clinical trials for cancer vaccines. It is already used in vaccines against several infectious agents, including the hepatitis B virus.

Bacille Calmette-Guerin (BCG) is a bacterium that is related to the bacterium that causes tuberculosis. The effect of BCG infection on the immune system makes this bacterium useful as a form of anticancer immunotherapy. BCG was one of the earliest immunotherapies used against cancer. It is FDA approved as a routine treatment for superficial bladder cancer. Its usefulness in other cancers as a nonspecific adjuvant is also being tested. Researchers are looking at injecting BCG to give an added boost to the immune system when using chemotherapy, radiation therapy, or other types of immunotherapy.

Incomplete Freund's Adjuvant (IFA) is given together with some experimental therapies to help stimulate the immune system and to increase the immune response to cancer vaccines. IFA is a liquid consisting of an emulsifier in white mineral oil.

QS-21 is a relatively new immune stimulant made from a plant extract that increases the immune response to vaccines used against melanoma.

DETOX is another relatively new adjuvant. It is made from parts of the cell walls of bacteria and a kind of fat. It is used with various immunotherapies to stimulate the immune system.

Keyhole limpet hemocyanin (KLH) is another adjuvant used to boost the effectiveness of cancer vaccine therapies. It is extracted from a type of sea mollusc.

Dinitrophenyl (DNP) is a hapten/small molecule that can attach to tumor antigens and cause an enhanced immune response. It is used to modify tumor cells in certain cancer vaccines.

The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Nasal sprays may be useful formulations.

Preferred unit dosage formulations are those containing a single or daily dose or unit, daily sub-dose or an appropriate fraction thereof, of an active ingredient.

It should be understood that in addition to the ingredients particularly mentioned above, the formulations of this invention may include other agents conventional in the art having regard to the type of formulation in question, for example those suitable for oral administration may include flavouring agents.

It will be appreciated that the therapeutic molecule can be delivered to the locus by any means appropriate for localised administration of a drug. For example, a solution of the therapeutic molecule can be injected directly to the site or can be delivered by infusion using an infusion pump. The construct, for example, also can be incorporated into an implantable device which when placed at the desired site, permits the construct to be released into the surrounding locus.

The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic^{R}.

Conveniently, the nucleic acid vaccine may comprise any suitable nucleic acid delivery means, as noted above. The nucleic acid, preferably DNA, may be naked (ie with substantially no other components to be administered) or it may be delivered in a liposome or as part of a viral vector delivery system.

The subject may be administered a combination of polypeptides and polynucleotides, as discussed above.

By “effective amount” we include the meaning that sufficient quantities of the agent are provided to produce a desired pharmaceutical effect beneficial to the health of the recipient.

All documents referred to herein are, for the avoidance of doubt, hereby incorporated by reference.

The invention is now described by reference to the following, non-limiting, figures and examples.

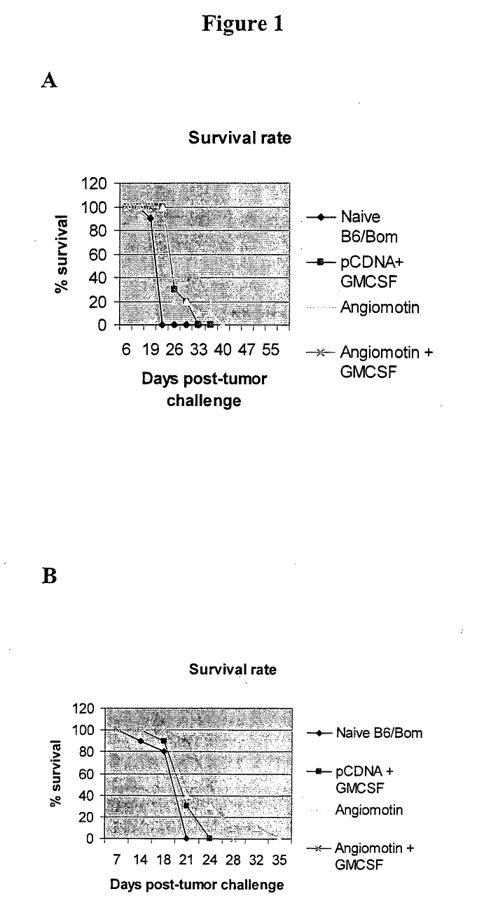

FIG. 1.

Mice were vaccinated with the angiomotin vaccine plasmid, or a control vector with or without the GM-CSF plasmid. A booster vaccination was given 14 days later. 2 weeks after the second vaccination mice were challenged with 100,000 live, B16 tumor cells administered intraperitoneally. Tumor growth was measured over 3 weeks. A and B represent two separate experiments.

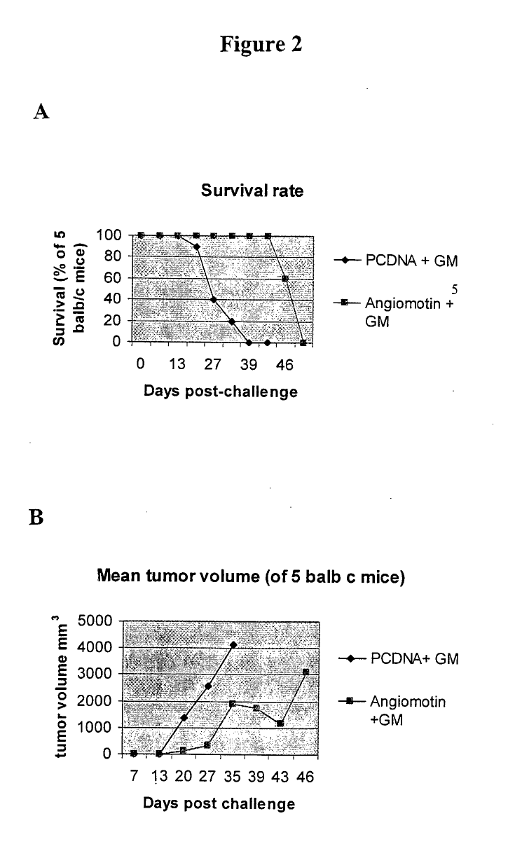

FIG. 2.

Mice were vaccinated with the angiomotin vaccine or a control plasmid with the GM-CSF plasmid. A booster vaccination was given 14 days later. 2 weeks after the second vaccination mice were challenged with 100,000 live, D2F2 tumor cells administered intraperitoneally. Tumor growth was measured over 3 weeks. A represents the survival of the mice whereas B represents the mean tumor volume.

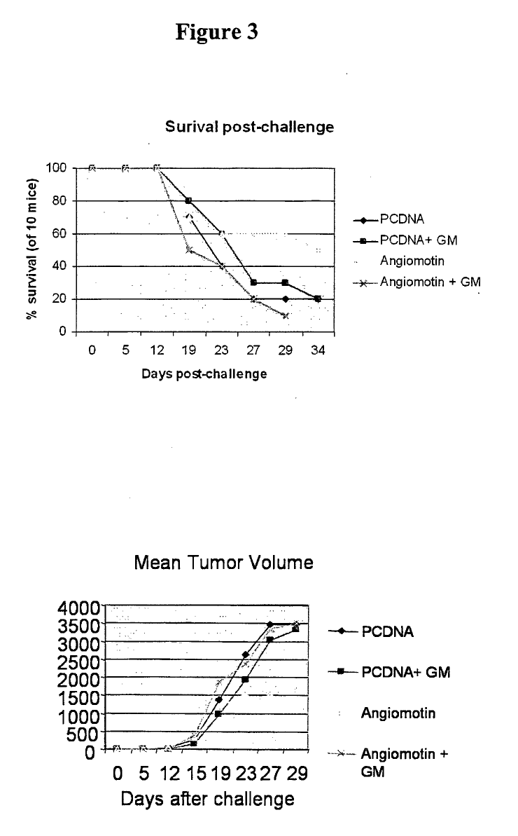

FIG. 3

Mice were vaccinated with the angiomotin vaccine or a control plasmid with the GM-CSF plasmid. A booster vaccination was given 14 days later. 2 weeks after the second vaccination mice were challenged with 60,000 live, EL4 tumor cells administered intraperitoneally. Tumor growth was measured over 3 weeks. A represents the survival of the mice whereas B represents the mean tumor volume.

FIG. 4

Amino acid and nucleotide sequences of human angiomotin.

FIG. 5

Anti-angiomotin vaccination. Angiomotin as a single therapeutic agent. BALB-neuT mice, which spontaneously develop tumors as a result of overexpressing the transforming rat Her.2/neu oncogene under the control of the mouse mammary tumor virus promoter (Boggio et al, J. Exp. Med 1998, 188;589-96), were vaccinated at the age of 6 weeks with the angiomotin vaccine plasmid, or a control vector, or left untreated. The mice were vaccinated by the method of “electroporation” as described in Quaglino et al (Cancer Research 64, 2858-64, 2004). Briefly, a total of 25 microgram of the EC-TM plasmids were injected into the tibial muscle of the anesthetized mice. Electric pulses were applied by two electrodes placed on the shaved skin covered with a conducting gel. Two square wave 25 ms, 375 V/cm pulses were generated by a T820 electroporator (BTX, San Diego, Calif.). A booster vaccination was given 2 week later. The percentage of tumor-free mice in each group of mice is shown.

FIG. 6

Two component therapy: Amot.

BALB-neuT mice were vaccinated at 10 weeks of age with the angiomotin vaccine plasmid, or a control vector, or left untreated. A further population of mice were vaccinated with angiomotin vaccine plasmid and TMEC, a tumor antigen. A booster vaccination was given 2 weeks later. The percentage of tumor-free mice in each population is shown.

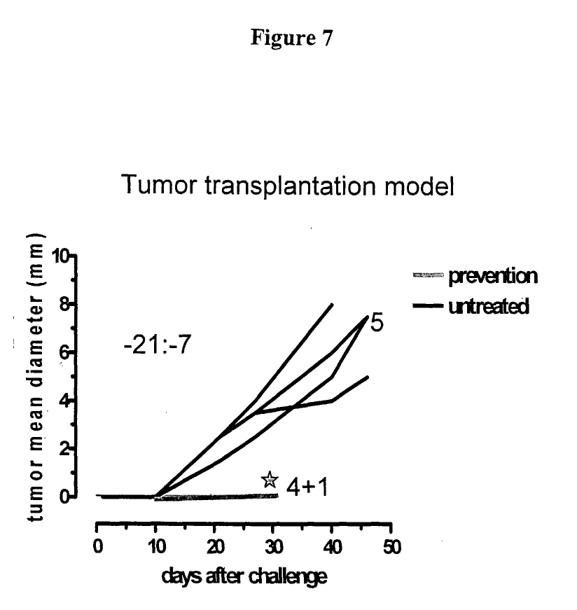

FIG. 7

Tumour transplant model

Balb/c mice were vaccinated at day −21 and −7 before s.q. injection of the TUBO breast cancer cell-line. The tumor mean diameter is shown.

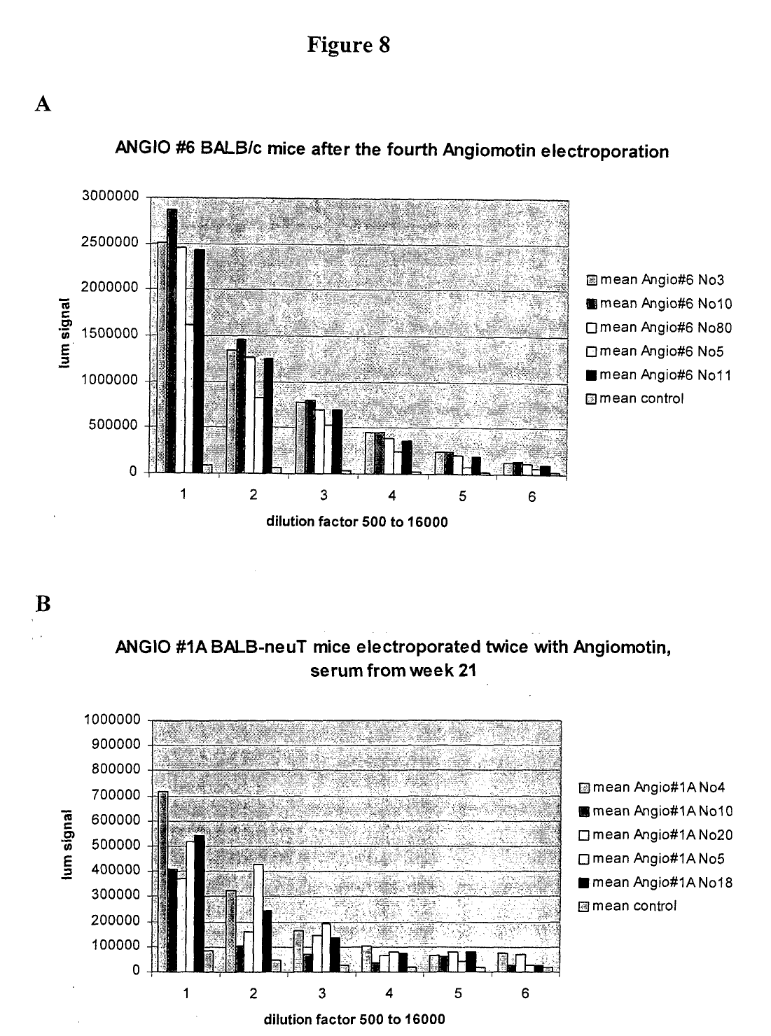

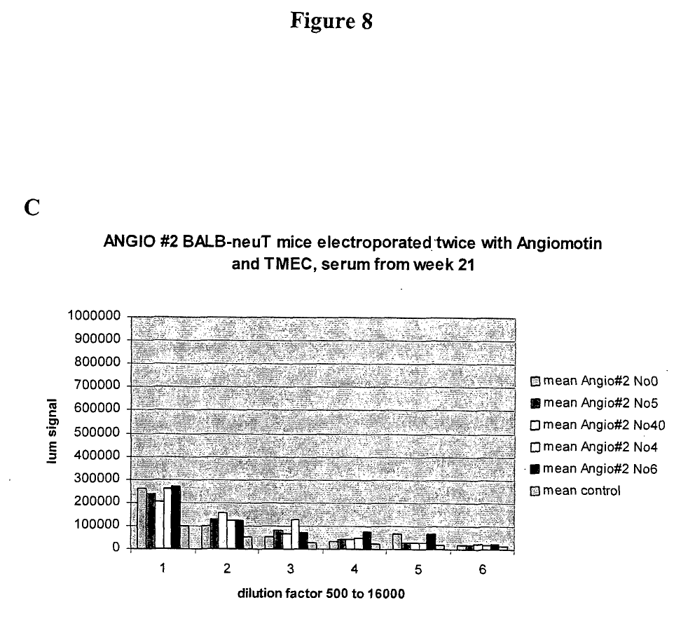

FIG. 8

Antibody response against mouse angiomotin.

The antibody response against murine Angiomotin, in sera from Angiomotin vaccinated mice, was measured in an ELISA (FIG. 8). The lum signal on the y-axis represents the amount of Angiomotin specific IgG antibodies and the numbers 1-6 on the x-axis represents different dilutions (500-16000) of the sera. Sera from the following mice were analysed:

A) Angio #6, mouse No 3, 5, 10, 11, 80; BALB/c mice electroporated four times, every second week, with Angiomotin vaccin plasmid. The sera taken after the fourth electroporation were analysed and compared to control mice, which had been electroporated at week 10 and 12with TMEC alone.

B) Angio #1A, mouse No 4, 5, 10, 18, 20; BALB-neuT mice electroporated at week 10 and 12 with Angiomotin vaccin plasmid. Sera taken week 21 were analysed and compared to control mice, which had been electroporated at week 10 and 12 with TMEC alone.

C) Angio #2, mouse No 0, 4, 5, 6, 40: BALB-neuT mice electroporated at week 10 and 12 with Angiomotin and TMEC vaccine plasmids. Sera taken week 21 were analysed and compared to control mice, which had been electroporated at week 10 and 12 with TMEC alone.

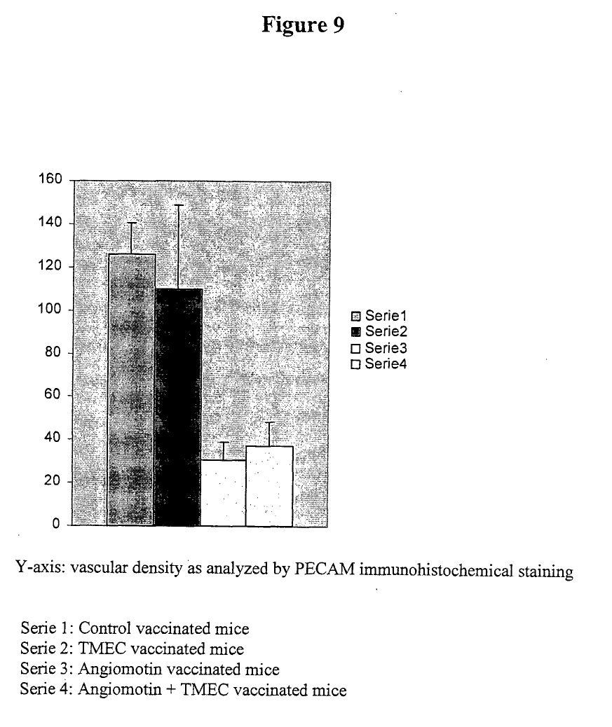

FIG. 9: Anti-angiomotin DNA vaccination inhibits angiogenesis in vivo.

BALBc mice were vaccinated with angiomotin alone or in combination with TMEC. The mice were injected with matrigel containing 200 ng/ml basic fibroblast growth factor two weeks after the last vaccination. The matrigel plugs were harvested seven days later and vascular density was quantified as described by Weidner et al N Engl J Med. 1991 Jan. 3;324(1):1-8.

### MATERIALS METHODS AND EXAMPLES

### Materials

C57B1 and Balb/c mice were obtained from the animal unit at the Microbiology and Tumor Biology Center, Karolinska institute.

The murine tumor cell lines used for the evaluation included the mouse mammary carcinoma D2F2 from Dr. Wei Zen Wei, Karmanos Cancer Center, the EL-4 lymphoma from the MTC research unit and the B16 melanoma line from Dr. I. J. Fidler, MD Anderson Cancer Center, Houston, TC.

### Example 1

### Preparing of a DNA Vaccine Encoding Angiomotin

Angiomotin-expressing plasmid was constructed by inserting the full-length cDNA coding for the angiomotin molecule into the pCDNA3 vector as per the instructions of the commercial vendor for the vector (Invitrogen Inc.). This vector is designed for protein expression in mammalian cells and contains a CMV promoter (U.S. Pat. Nos. 5,168,062 and 5,385,839; University of Iowa Research Foundation), Multiple cloning site, bovine growth hormone polyadenylation sequence (U.S. Pat. No. 5,122,458) and a neomycin resistance gene for selection of stable cell-lines.

The full-length cDNA of Angiomotin was inserted into the pCDNA3 vector. The orientation and identity of the resulting plasmid was confirmed with restriction mapping. Transfection of the pCDNA3 resulted in angiomotin expression as analyzed by western blot.

### Example 2

### Vaccination of Mice with a DNA Vaccine Encoding Angiomotin

Balb/c mice (6 mice per group) (FIG. 1) or C57B1 mice (6 mice per group) (FIGS. 2 and 3) were vaccinated twice with 2 weeks interval with the above described angiomotin pDNA construct using gene gun immunization, or as control the same pcDNA “empty” control vector (PCDNA).

The plasmid is prepared according to Qiagen standard protocol (Qiagen endotoxin free plasmid kit, WWR International). The plasmid is dissolved and kept in water at a concentration of 1 mg/ml. From this stock, the plasmid is mixed with gold in 99.5% ethanol and coated on to a plastic tube. Shots are prepared from the DNA-gold mix, and delivered intra-epidermally in the region of the inguinal lymph nodes, using the Helios gene gun (Biorad, Stockholm). Each mouse received two shots at different locations at a concentration of 0.6-1.0 μg DNA per shot. This procedure was repeated twice with a 14 days interval. The procedure was the same for all plasmids used, including the GM-CSF plasmid discussed below.

In some groups of mice, a plasmid coding for the cytokine GM-CSF was mixed with the angiomotin coding plasmid (Angiomotin+GM) or mixed with the control vector (PCDNA+GM) in equal molar quantities and administered by gene-gun as detailed above. The GM-CSF expressing plasmid has been described elsewhere. (Charo, J. et al. J. Immunol, 163: 5913-5919, 19991).

### Example 3

### Evaluation of Tumor Growth and Survival of Vaccinated Mice

Two weeks after the last immunization, the mice were challenged with about 5×10 4 live B16 cells (FIG. 1), D2F2 melanoma cells (FIG. 2) or EL4 lymphoma cells (FIG. 3). Groups of mice were inspected twice every week for number of survivors in each group and for mean tumor volume, as measured by a micrometer.

There are a number of animal studies which demonstrate that a vaccine candidate may serve both prophylactically (ie to prevent tumors in an animal challenged with tumor cells) and therapeutically (ie administration of the vaccine causes regression of previously established tumors), for example Cavallo et al (1993) Protective and curative potential of vaccination with IL-2 gene-transfected cells from a spontaneous mouse mammary adenocarcinoma. Cancer Res 21:5067; Nanni et al (2001) Combined allogeneic tumor cell vaccination and systemic interleukin-12 prevents mammary carcinogenesis in HER-2/neu transgenic mice. J Exp Med 194:1195.

### Example 4

### Prediction of Peptide Epitopes of Angiomotin That May be of Significance in Generating Angiomotin-Specific Immune Responses

The immunogenicity of various peptides derivatives of a protein are known to be restricted in any individual, depending on the tissue antigens (HLA) or the particular individual. Using established computerized algorithms (http://www.bimas.dcrt.nih.gov/molbio/hla_bind/index.html), the binding affinity and predictive value of various peptide motifs derived from angiomotin and restricted to various HLA antigens was established. The predicted peptides that are potentially immunogenic and restricted to the HLA A201 epitope are provided in Table 1. HLA A201 occur at a frequency of approximately 65% in the Caucasian population. Similar peptide motifs with potential value for immunotherapy can also be predicted for other HLA alleles.

HLA A201 is an allele important for vaccinating Caucasian population. Another important allele is HLA A2402 which is present on approximately 60% of the asian population.

TABLE 1
Scoring Results

| | | | Score (Estimate of Half Time of |
|---|---|---|---|
| | Start | Subsequence | Disassociation of a Molecule |
| Rank | Position | Residue Listing | Containing This Subsequence) |
| 1 | 196 | KMQQALVQL | 124.199 |
| 2 | 388 | GLLSHSSTL | 79.041 |
| 3 | 169 | ALSNAQAKV | 69.552 |
| 4 | 285 | FALDAAATV | 67.409 |
| 5 | 94 | RMHDFNRDL | 48.075 |
| 6 | 417 | ILLGGDYRA | 31.249 |
| 7 | 31 | ILSDENRNL | 29.779 |
| 8 | 427 | YVPSTPSPV | 27.995 |
| 9 | 325 | ILMANKRCL | 26.874 |
| 10 | 389 | LLSHSSTLT | 12.668 |
| 11 | 52 | RLQKVETEI | 10.433 |
| 12 | 21 | IVSRAQQMV | 10.346 |
| 13 | 498 | MVAAAPVAV | 10.346 |
| 14 | 536 | GQIPAAASV | 7.052 |
| 15 | 262 | ILALEADMT | 6.208 |
| 16 | 345 | QIIEKDAMI | 5.881 |
| 17 | 62 | RVSEAYENL | 5.633 |
| 18 | 574 | ALVPVPAPA | 4.968 |
| 19 | 200 | ALVQLQAAC | 4.968 |
| 20 | 630 | RLSIPSLTC | 4.968 |

### Example 5

### Angiomotin Vaccination as Antiangiogenic Therapy

The previous examples demonstrate that Angiomotin vaccination can provide prophylactic protection against tumourgenesis. We now provide data demonstrating that Angiomotin vaccination may be used as part of angiogenesis therapy.

### Method

The BALB-neuT mouse is a transgene breast cancer mouse model. BALB-neuT mice spontaneously develop tumors as a result of overexpressing the transforming rat Her.2/neu oncogene under the control of the mouse mammary tumor virus promoter (Ref Boggio et al, J. Exp. Med 1998, 188;589-96).

BALB-neuT mice (5 per group) were vaccinated twice at the ages indicated by the arrows in FIGS. 5 and 6 with 25 μg of plasmid by electroporation into the tibialis anterior using a T820 Electrosquare porator using pulse parameters of 25 msec at 375 V/cm. The plasmid was prepared for electroporation using the protocol described in Example 2 above.

### Results and Discussion

In FIG. 5, three groups of mice were vaccinated at 6 and 8 weeks with empty vector control plasmid (pcDNA3), using the electroporation method described above; Angiomotin plasmid; or untreated (plasmids as described in Example 1 above). As can be seen, 50% of mice treated with Angiomotin plasmid were tumour free after 30 weeks, while all of the untreated or control treated mice had tumours after 27 weeks. Therefore Angiomotin can act to prevent tumour development in this mouse strain.

In FIG. 6, four groups of mice were vaccinated at 10 and 12 weeks of age with empty vector control plasmid (pcDNA3); TMEC; TMEC+Angiomotin plasmid; or untreated. TMEC is the p185^{neu} TM-ECD plasmid which encodes “transmembrane extra cellular” part of the p185 neu, a fragment of the oncogene responsible for tumourgenesis in BALB-neuT mice (Boggio et al, J. Exp. Med 1998, 188;589-96).

As can be seen, while all of the untreated or control treated mice had tumours after 27 weeks, 40% of the mice treated with TMEC plasmid alone were tumour free after 50 weeks. Moreover, 100% of the mice treated with both TMEC plasmid and Angiomotin plasmid were tumour free after 50 weeks.

Therefore dual TMEC/Angiomotin plasmid vaccination has a greater therapeutic effect in preventing tumour development than TMEC or Angiomotin vaccination alone. Since the mammary glands of virgin BALB-neu T mice at the age of vaccination (week 10 and 12) already have multiple in situ carcinomas (see references given above), this demonstrates that this combined vaccination with angiomotin and tumor vaccine has a therapeutic effect and can halt further tumor development.

In FIG. 7. Adult Balb/c mice were vaccinated with the Angiomotin pDNA vaccine described above, using the electroporation method, also described above, 21 and 7 days before subcutaneous (s.q.) injection of TUBO breast cancer cells. The TUBO breast cancer cell is a transplantable cell line isolated from a tumor derived from the BALB-neu T mice described above. As can be seen, 4 of those mice vaccinated with the Angiomotin pDNA vaccine (marked “prevention”) were completely tumor free, while 1 mouse had a 1 mm large tumor. All untreated mice developed large tumors. Therefore vaccination with Angiomotin as a “monotherapy” can act as a prophylactic vaccine in mice against tumor development arising from this breast cancer line.

The data presented in FIGS. 5 to 7 demonstrate that Angiomotin plasmid vaccination as a “monotherapy” in a transplantable tumor model can prevent tumor growth and, in a spontaneous transgenic Her2/neu breast cancer model, can markedly reduce tumor development. Moreover, the data in FIG. 6 show that combined Angiomotin plasmid/tumour antigen vaccine administered to mice which already have developed multiple in situ preneoplastic lesions can confer total tumor protection and has a greater effect than either the Angiomotin plasmid or tumour antigen alone, demonstrating that Angiomotin and tumour antigen based vaccines can interact synergistically in a therapeutic tumor model. This strongly suggests that angiomotin as a monotherapy or in a combined vaccine may be of use in the therapy of human tumors or in angiogenesis therapy of other diseases.

### Example 6

### Angiomotin Vaccination Induces Anti-Angiomotin Antibody Production

Groups of 12 weeks old BALB-neuT (FIG. 8 panels B and C) or BALB/c mice (FIG. 8 Panel A) mice were immunized by electroporation with human angiomotin pDNA, as described above, four times (Panel A) or twice (Panel B) with 2 weeks interval. Panel C shows results from mice immunized by electroporation with angiomotin pDNA combined with TMEC pDNA. At week 21, mice were bled through the tail vein and serum collected and frozen. Serum samples at the dilution factor from 500-16,000 from individual mice were tested in an ELISA assay specific for mouse angiomotin antibodies.

Murine Angiomotin was extracted from Angiomotin transfected Mouse Aortic Endothelial cells using polyclonal antibodies towards the C-terminus of Angiomotin.

The results demonstrates that mice, particularly after 4 times immunization with human angiomotin pDNA as a monotherapy, but also after 2 times immunization with this plasmid in combination with a TMEC tumor antigen (panel C), have developed high tittered antibodies to mouse angiomotin. This results show that immunization with human angiomotin pDNA electroporation can break the immunological tolerance to the mouse angiomotin molecule, and also suggests, although does not prove, that these antibodies could be the active mechanism behind the anti-angiogenic effect and the anti-tumor effect in the immunized mice.

### Example 7

### Anti-Angiomotin DNA Vaccination Inhibits Angiogenesis In Vivo

5 BALBc mice were vaccinated with angiomotin alone or in combination with TMEC at −28 days, −14 days, i.e. 4 and 2 weeks, respectively, before start of the matrigel experiment. The angiomotin vaccine used was the pcDNA plasmid described previously in other examples.

The mice were injected with matrigel containing 200 ng/ml basic fibroblast growth factor two weeks after the last vaccination. The matrigel plugs were harvested seven days later and were fixed in paraformaldehyde and vessel infiltration was visualized by immunohistochemical staining with a rat monoclonal anti mouse PECAM antibody. Vascular density was quantified as described by Weidner et al N Engl J Med. 1991 Jan. 3;324(1):1-8. As can be seen from FIG. 9, angiogenesis was inhibited in mice vaccinated with angiomotin DNA.

There are a variety of adaptation and alterations of the embodiments described above which can be brought about without deviating from the spirit and scope of the invention. It is to be understood that no limitations with respect to the specific embodiments illustrated herein are intended or should be inferred. It is, of course, intended to cover by the appended claims all such modifications as fall within the scope of the claims.

1. The use of an angiomotin molecule or polynucleotide encoding an angiomotin molecule in the manufacture of a vaccine for vaccinating a subject with or at risk of an angiogenesis-related disease or disorder.

2. A method for treating a subject with or at risk of an angiogenesis-related disease or disorder, the method comprising the step of vaccinating the subject using a vaccine comprising an angiomotin molecule or polynucleotide encoding an angiomotin molecule.

3. The use or method of claim 1 or 2 wherein the angiogenesis-related disease or disorder is cancer, a solid tumour, hemangioma, ocular neovascularisation, diabetic retinopathy, macular degeneration, rheumatoid arthritis, inflammatory conditions (such as psoriasis, chronic inflammation of the intestines, asthma) or endometriosis.

4. A vaccine effective against blood vessel formation, comprising an effective amount of an angiomotin molecule or polynucleotide encoding an angiomotin molecule.

5. A method of eliciting an immune response against angiomotin by administering a vaccine comprising an angiomotin molecule or polynucleotide encoding an angiomotin molecule to a human.

6. The use, method or vaccine according to any one of the preceding claims wherein the angiomotin molecule is full length human angiomotin.

7. The use, method or vaccine according to any one of the preceding claims wherein the angiomotin molecule is a fragment of human angiomotin.

8. The use, method or vaccine according to claim 7 wherein the fragment of human angiomotin is a fragment of 9, 10, 11 or 12 amino acids in length.

9. The use, method or vaccine according to claim 8 wherein the fragment is a fragment as listed in Table 1.

10. The use, method or vaccine of any of the preceding claims wherein the vaccine further comprises as antigen a tumour antigen and/or a angiogenic factor and/or one or more antibodies against a tumor antigen or antigenic factor.

11. The use, method or vaccine of any one of the preceding claims wherein the vaccine further comprises an immunostimulatory molecule.

12. The use, method or vaccine of claim 11 wherein the immunostimulatory molecule is a cytokine or polynucleotide encoding a cytokine.

13. The use, method or vaccine of any one of the preceding claims wherein the vaccine comprises a cell or cell extract.

14. The use, method or vaccine of claim 13 wherein the cell is an antigen presenting cell which is loaded with the angiomotin molecule or transfected with polynucleotide that encodes an angiomotin molecule.

15. The use, method or vaccine of claim 13 wherein the cell is a tumour cell expressing angiomotin or an endothelial cell expressing angiomotin.

16. The use or method of any one of claims 1 to 3, 5 to 10 wherein the vaccine is administered to the patient by ex vivo administration of the vaccine to cells from the patient, followed by transfer of stimulated immune cells back into the patient.

17. A method of generating an immune response against angiomotin in a mammal, the method comprising the steps of (i) stimulating ex vivo immune cells collected from the mammal with an angiomotin molecule or polynucleotide encoding an angiomotin molecule, (ii) transferring the stimulated immune cells back into the mammal, such that transfer of the cells back into the said mammal generates an immune response against angiomotin.

18. Use of an angiomotin molecule or polynucleotide encoding an angiomotin molecule in the manufacture of a medicament for generating an immune response against angiomotin in a mammal using a method comprising the steps of (i) stimulating ex vivo immune cells collected from the mammal with the medicament, (ii) transferring the stimulated immune cells back into the mammal, such that transfer of the cells back into the said mammal generates an immune response against angiomotin.

19. The method of claim 17 or use of claim 18 wherein the mammal is a human.

20. The method of claim of 17 or 19 or use of claim 18 or 19 wherein the elicited immune response serves prophylactically or therapeutically to inhibit the onset or progress of an angiogenesis-related disease.

21. The method of any one of claims 17, 19 or 20 or use of any one of claims 18 to 20 wherein the elicited immune response serves to prophylactically or therapeutically inhibit the onset or progress of a malignant disease.
